(19) United States
(12) Patent Application Publication
(10) Pub. No.: US 2015/0116125 A1
(43) Pub. Date: Apr. 30, 2015

(54) WRISTBAND WITH REMOVABLE ACTIVITY MONITORING DEVICE

(71) Applicant: JayBird LLC, Salt Lake City, UT (US)

(72) Inventors: JUDD ARMSTRONG, Salt Lake City, UT (US); STEPHEN DUDDY, Salt Lake City, UT (US)

(73) Assignee: JayBird LLC, Salt Lake City, UT (US)

(21) Appl. No.: 14/062,815

(22) Filed: Oct. 24, 2013

(51) Int. Cl. A61B 5/00; A61B 5/0205; A61B 5/1455; A61B 5/0456; A61B 5/11

(52) U.S. Cl. CPC A61B 5/0006; A61B 5/0456; A61B 5/1112; A61B 5/681; A61B 5/14551; A61B 5/0205; A61B 5/002; A61B 5/0022; A61B 5/02405
USPC 340/870.01

(57) ABSTRACT

A wearable wristband with a removable device comprises an electronic capsule and a wristband, wherein the electronic capsule comprises a wrist biosensor, a finger biosensor, a watertight casing, a battery, one or more circuit boards electronically coupled to the wrist biosensor and a finger biosensor. The wristband comprises a flexible material notched with a cavity and an aperture, wherein the cavity is shaped to receive the electronic capsule and the aperture shaped to receive the finger biosensor.

### FIELD OF THE INVENTION

The present disclosure is generally related to activity monitoring devices, and more specifically towards a wristband with a removable activity monitoring device.

### BACKGROUND OF THE INVENTION

Wearable activity monitoring devices are growing in popularity. The market includes wearable devices that measure the number of steps the wearer takes, or the wearer's heart rate. Many such devices interface with computer software to allow visualization of the recorded data. However, despite this demand, the current consumer market for wearable activity monitoring devices mostly comprises products with limited functionality. For example, some devices use accelerometers to measure steps taken while walking or running, while other distinct devices can measure heart rate. However, very few devices perform multiple simultaneous functions. In addition, none of these devices are configured to automatically detect the activity being performed, measure quality of sleep, and measure a cardiac recovery score. Moreover, none of these devices includes all of this data in the computer program interfaced with the device to inform the calculation of athletic activity goals, plans, and strategies. Devices that are capable of measuring a cardiac recovery score do not perform automatic activity detection. Such devices also require wearing external sensors on the chest, as opposed to incorporating the sensors into the wearable device itself. In addition, currently available wearable athletic monitoring devices require a sensor to be inserted into the wearable element of the device—usually a wristband—using bulky mechanical attachments such as latches or clips. These devices are uncomfortable to wear, and thus undesirable for 24-hour per day use.

### BRIEF SUMMARY OF EMBODIMENTS OF THE INVENTION

The present disclosure is directed towards an activity monitoring device. In particular, embodiments of the invention provide a wearable athletic monitoring device comprised of a comfortable wristband (e.g. silicone) and a removable electronic capsule designed to be affixed to the wristband (e.g. with magnets). Further, the device is configured to automatically detect the activity being performed using an accelerometer and/or other incorporated sensors, and also comprises one or more biosensors for measuring cardio-electric signals and calculating cardiac recovery scores.

Further, because the electronic capsule is easily removable from the wristband, users can easily swap out wristbands of one size, material composition, and color for a wristband of another size, material, and material composition to provide a more comfortable, cost-effective, or convenient wearing experience for the user. Alternatively, the electronic module may easily be affixed to other wearable structures, such as shoes or socks.

In one embodiment of the disclosure, an activity monitoring device comprises: an electronic capsule and a wristband; wherein the electronic capsule comprises a wrist biosensor, a finger biosensor, a battery, one or more circuit boards, and a casing. The finger biosensor protrudes outward from a first side of the casing and is electronically coupled to the one or more circuit boards, the wrist biosensor protrudes outward from a second side of the casing and is electronically coupled to the one or more circuit boards, the battery is enclosed inside of the casing and is electronically coupled to the one or more circuit boards, and the one or more circuit boards are enclosed inside of the casing. The wristband comprises a flexible material sized to encircle a human wrist wherein a cavity is notched on the radially inward facing side of the wristband and shaped to substantially the same dimensions as the profile of the electronic capsule, and an aperture is located in the flexible material within the cavity and is shaped to substantially the same dimensions as the profile of the finger biosensor such that the finger biosensor protrudes through the aperture when the electronic capsule is positioned inside of the cavity.

In some embodiments of the disclosure, the flexible material of the wristband may comprise silicone. Alternatively, the flexible material may comprise plastic, metal links, composite material, rubber, leather, fabric, or other flexible materials.

In some embodiments of the disclosure, the electronic capsule may further comprise an accelerometer. Other sensors may also be incorporated in the electronic capsule, such as optical heart-rate sensors and other health or activity monitoring sensors. In addition, the electronic capsule may also comprise a wireless transmitter such as a Bluetooth transmitter, a Wi-Fi transmitter, a GPS transmitter, a multi-purpose transmitter, or other transmitters apparent to a person of ordinary skill in the art. In many of these embodiments, the electronic capsule casing may comprise molded plastic, metal, composite, or other moldable materials.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present disclosure, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention.

FIG. 1 illustrates a cross-sectional view of the wristband and electronic modules of an example activity monitoring device.

FIG. 2 illustrates a perspective view of an example activity monitoring device.

FIG. 3 illustrates a cross-sectional view of an example assembled activity monitoring device.

FIG. 4 illustrates a side view of an example electronic capsule.

FIG. 5 illustrates a cross-sectional view of an example electronic capsule.

FIG. 6 illustrates perspective views of wristbands as used in one embodiment of the disclosed activity monitoring device.

These figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION OF THE EMBODIMENTS OF THE INVENTION

The present disclosure is directed towards an activity monitoring device, and more specifically towards a wristband with a removable activity monitoring device. According to some embodiments of the disclosure, the activity monitoring device comprises a wrist band and a removable electronic capsule.

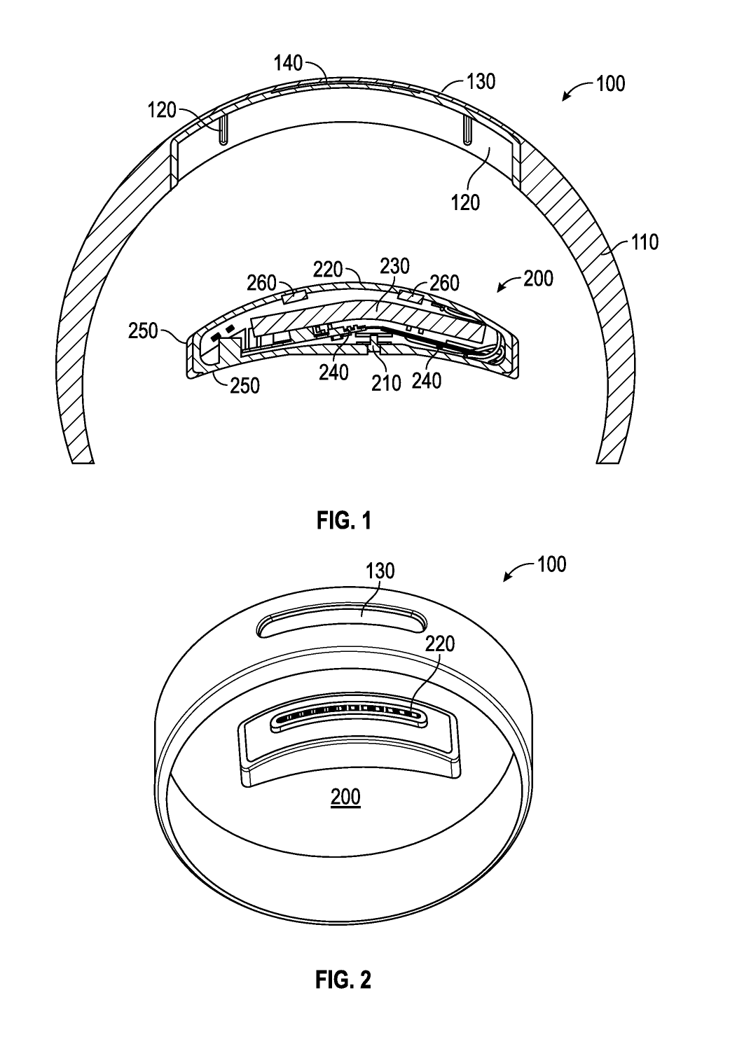

FIG. 1 is a diagram illustrating a cross-sectional view of an exemplary embodiment of an activity monitoring device. Referring now to FIG. 1, an activity monitoring device comprises an electronic capsule 200 and a wristband 100. The electronic capsule 200 comprises a wrist biosensor 210, a finger biosensor 220, a battery 230, one or more logic circuits 240, and a casing 250. By way of example, the logic circuits may be printed circuit boards (PCB's). Finger biosensor 220 protrudes outward from a first side of casing 250, and wrist biosensor 210 protrudes outward from a second side of casing 250. The battery 230 and logic circuits 240 are enclosed inside of the casing 250. In some embodiments, casing 250 is molded plastic or another moldable material. Additionally, the casing may be sealed using an ultrasonic welding process such that it is watertight. Battery 230 is electronically coupled and supplies power to the logic circuits 240.

In some embodiments, the one or more logic circuits 240 comprise an accelerometer, a wireless transmitter, and circuitry. The logic circuits may further comprise a gyroscope. These logic circuits may be configured to process electronic input signals from the biosensors and the accelerometer, store the processed signals as data, and output the data using the wireless transmitter. The transmitter is configured to communicate using available wireless communications standards. For example, in some embodiments, the wireless transmitter may be a BLUETOOTH transmitter, a Wi-Fi transmitter, a GPS transmitter, a cellular transmitter, or some combination thereof. In an alternative embodiment, the wireless transmitter may further comprise a wired interface (e.g. USB, fiber optic, HDMI, etc.) for communicating stored data.

The logic circuits 240 are electrically coupled to the wrist biosensor 210 and the finger biosensor 220. In addition, the logic circuits are configured to receive and process a plurality of electric signals from each of the wrist biosensor 210 and finger biosensor 220. In some embodiments, the plurality of electric signals comprise an activation time signal and a recovery time signal such that the logic circuits 240 may process the plurality of signals to calculate an activation recovery interval equal to the difference between the activation time signal and the recovery time signal. In some embodiments, the plurality of signals may comprise electro-cardio signals from a heart, and the logic circuits may process the electro-cardio signals to calculate and store a RR-interval, and the RR-interval may be used to calculate and store a heart rate variability (HRV) value. Here, the RR-interval is equal to the delta in time between two R-waves, where the R-waves are the electro-cardio signals generated by a ventricle contraction in the heart.

In some embodiments, the logic circuits may further detect and store metrics such as the amount of physical activity, sleep, or rest over a recent period of time, or the amount of time without physical activity over a recent period of time. The logic circuits may then use the HRV, or the HRV in combination with said metrics, to calculate a recovery score. For example, the logic circuits may detect the amount of physical activity and the amount of sleep a user experienced over the last 48 hours, combine those metrics with the user's HRV, and calculate a recovery score of between 1 and 10, wherein the recovery score could indicate the user's physical condition and aptitude for further physical activity that day. The recovery score may also be calculated on a scale of between 1 and 100, or any other scale or range.

In some embodiments, the finger biosensor and wrist biosensor may be replaced or supplemented by a single biosensor. In one such embodiment, the single biosensor is an optical biosensor such as a pulse oximeter configured to detect blood oxygen saturation levels. The pulse oximeter may then output a signal to the logic circuits indicating detected a cardiac cycle phase, and the logic circuits may use cardiac cycle phase data to calculate a HRV value.

Wristband 100 comprises a material 110 configured to encircle a human wrist. In one embodiment, wristband 100 is adjustable. A cavity 120 is notched on the radially inward facing side of the wristband and shaped to substantially the same dimensions as the profile of the electronic capsule. In addition, an aperture 130 is located in the material 110 within cavity 120. The aperture 130 is shaped to substantially the same dimensions as the profile of the finger biosensor 220. The cavity and aperture combination is designed to detachably couple to the electric capsule 200 such that, when the electric capsule 200 is positioned inside cavity 120, the finger biosensor 220 protrudes through the aperture 130. Electronic capsule 200 may further comprise one or more magnets 260 configured to secure capsule 200 to cavity 120. Magnets 260 may be concealed in casing 250. Alternatively, cavity 120 may be configured to conceal magnets 260 when electric capsule 200 detachably couples to the cavity and aperture combination.

Wristband 100 may further comprise a steel strip 140 concealed in material 110 within cavity 120. In this embodiment, when the electronic capsule 200 is positioned within the cavity 120, the one or more magnets 260 are attracted to the steel strip 140 and pull electronic capsule 200 radially outward with respect to the wristband. The force provided by magnets 260 may detachably secure electronic capsule 200 inside cavity 120. In alternative embodiments, the electronic capsule may be positioned inside the wristband cavity and affixed using a form-fit, press-fit, snap-fit, friction-fit, VELCRO, or other temporary adhesion or attachment technology.

FIG. 2 illustrates a perspective view of one embodiment of the disclosed activity monitoring device. In this embodiment, wristband 100 and electronic capsule 200 are unassembled. Finger biosensor 220 protrudes from the top of electronic capsule 200, and aperture 130 is substantially the same dimensions as the profile of finger biosensor 220. In one embodiment, material 110 may comprise silicone.

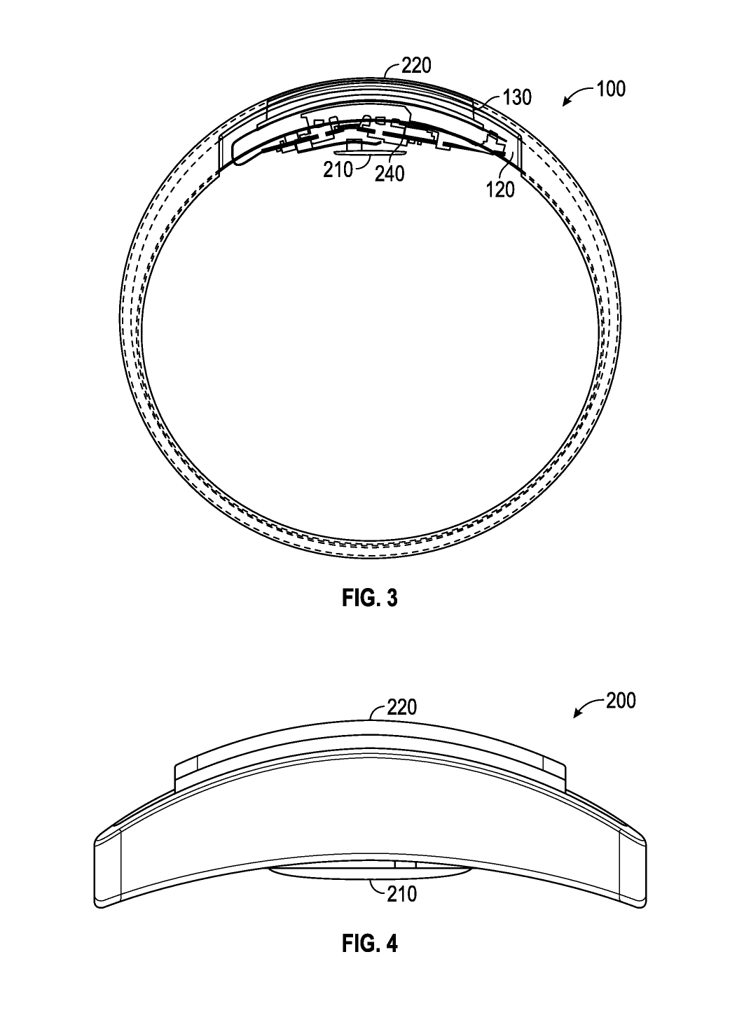

FIG. 3 illustrates a cross-sectional view of one embodiment of a fully assembled wristband with removable athletic monitoring device. In this embodiment, electronic capsule 200 is positioned inside cavity 120 of wristband 100 such that finger biosensor 220 protrudes through aperture 130. Wrist biosensor 210 protrudes from the radially inward facing side of the assembled device. In this configuration, wrist biosensor 210 may contact the skin on a human wrist when the wrist band is attached to human wrist.

FIG. 4 illustrates a side view of an electronic capsule 200 according to one embodiment of the invention. More particularly, finger biosensor 220 protrudes from a first side of the electronic capsule 200 and wrist biosensor 210 protrudes from a second side of electronic capsule 200. Casing 250 encloses components inside of electronic capsule 200. The casing 250 may comprise moldable plastic. Alternatively, casing 250 may comprise metal, rubber, composite material, or another moldable material. In one embodiment, casing 200 is ultrasonically welded together to make the casing water tight. In alternative embodiments, other methods may be used to make the casing water tight.

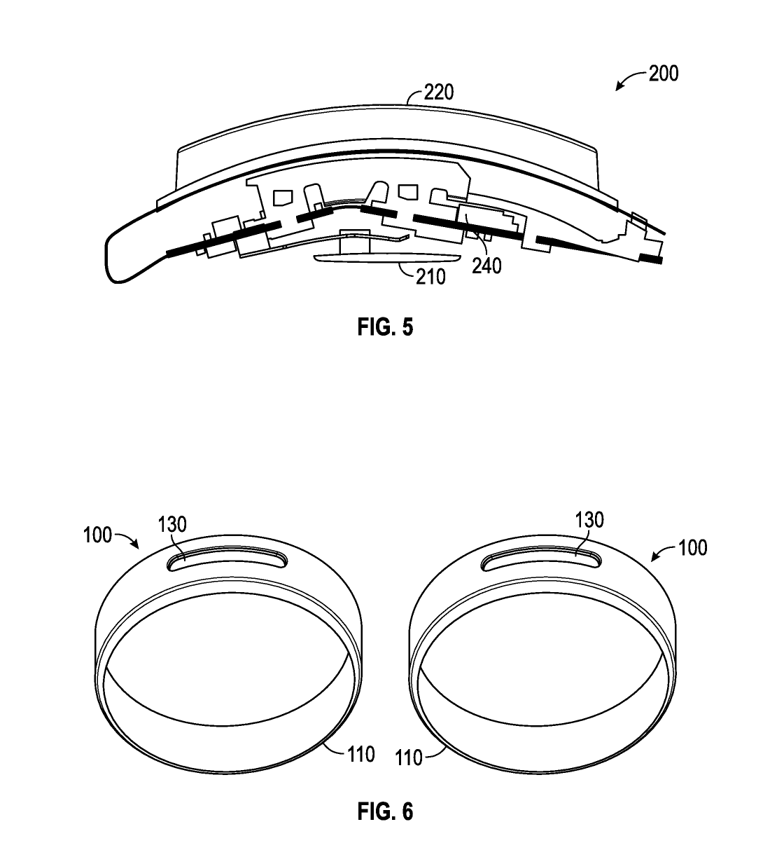

FIG. 5 illustrates a cross-sectional view of electronic capsule 200. In the illustrated embodiment, a finger biosensor 220 protrudes from a first side of the electronic capsule, and a wrist biosensor protrudes from a second side of the electronic capsule. Both the finger biosensor and the wrist biosensor are electronically coupled to the circuit boards 240.

FIG. 6 is a perspective view of two possible variants of the wristband according to some embodiments of the invention. Each wristband 100 comprises flexible material 110, and aperture 130 is disposed on each wristband 100. Each electronic capsule may be configured to standard dimensions such that it can be easily removed from one wristband and place in another wristband. Wristbands may be constructed with different dimensions, including different diameters, widths, and thicknesses, in order to accommodate different human wrist sizes and different preferences. In one embodiment, the wristbands may be adjustable to accommodate different human wrist sizes. Further, the wristbands may be made with different colors, and different flexible materials. In some embodiments, the flexible material 110 comprises silicone. Alternatively, the flexible material may comprise plastic, metal chain links, composite material, leather, synthetic leather, fabric, or other flexible materials.

In some embodiments of the invention, the electronic capsule may be detachably coupled to a cavity on a shoe and/or a sock. In other embodiments, the electronic capsule may be detachably coupled to sports equipment. For example, the electronic capsule may be detachably coupled to a skateboard, a bicycle, a helmet, a surfboard, a paddle boat, a body board, a hang glider, or other piece of sports equipment. In these embodiments, the electronic capsule may be affixed to the sports equipment using magnets. Alternatively, in other embodiments, the electronic capsule can be affixed using a form-fit, snap-fit, press-fit, friction-fit suction cup, VELCRO, or other technology that would be apparent to one of ordinary skill in the art.

In one embodiment of the invention, the electronic capsule may further comprise an optical sensor such as a heart rate sensor or oximeter. In this embodiment, the optical sensor may be positioned to face radially inward towards a human wrist when the wristband is fit on the human wrist. Alternatively, the optical sensor may be separate from the electronic capsule, but still detachably coupled to the wristband and electronically coupled to the circuit boards enclosed in the electronic capsule.

While various embodiments of the present invention have been described above, it should be understood that they have been presented by way of example only, and not of limitation Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

1. An activity monitoring device, comprising:
an electronic capsule, comprising:
a battery
a circuit board electronically coupled to the battery;
a casing enclosing the circuit board and the battery;
a finger biosensor electronically coupled to the circuit board protruding from a first side of the casing; and
a wrist biosensor electronically coupled to the circuit board protruding from a second side of the casing; and
one or more wristbands.

2. The activity monitoring device of claim 1, wherein each of the wristbands comprises a cavity notched on a radially inward side of the wristband that is shaped to substantially match a profile of the electronic capsule such that the electronic capsule is form-fit in place when positioned in the cavity.

3. The activity monitoring device of claim 2, wherein an aperture is located within the cavity, the aperture being shaped to substantially match a profile of the finger biosensor such that the finger biosensor protrudes through the aperture when the electronic capsule is positioned inside of the cavity.

4. The activity monitoring device of claim 3, wherein the one or more wristbands comprise wristbands of varying dimensions and wherein the electronic capsule is configured to interchange between the wristbands of varying dimensions.

5. The activity monitoring device of claim 4, wherein each wristband comprises silicone, rubber, leather, plastic, or metal.

6. The activity monitoring device of claim 3, wherein the electronic capsule further comprises one or more magnets concealed inside the casing and each of the wristbands further comprises a metal component located within the cavity, such that the one or more magnets are magnetically attracted to the metal component, thereby holding the electronic capsule in place when the electronic capsule is positioned in the cavity.

7. The activity monitoring device of claim 2, wherein the electronic capsule further comprises an accelerometer, a wireless transmitter, and one or more logic circuits to process electronic input signals from the finger biosensor, the wrist biosensor, and the accelerometer.

8. The activity monitoring device of claim 7, wherein the logic circuits store data from the finger biosensor, the wrist biosensor, and the accelerometer, and wherein the logic circuits transmit the data using the wireless transmitter.

9. The activity monitoring device of claim 8, wherein the finger biosensor and the wrist biosensor are each configured to detect a plurality of signals caused by electrical activity of the heart during one or more cardiac cycles, and the logic circuits are configured to process the plurality of signals and calculate an RR-interval.

10. The activity monitoring device of claim 9, wherein the logic circuits calculate a heart rate variability value and a recovery score.

11. The activity monitoring device of claim 7, wherein the wireless transmitter comprises a BLUETOOTH transmitter, a Wi-Fi transmitter, or a GPS transmitter.

12. The activity monitoring device of claim 1, wherein the casing of the electronic capsule is molded plastic.

13. The activity monitoring device of claim 12, wherein the molded plastic casing is ultrasonic welded together to achieve a watertight seal.

14. An electronic capsule for monitoring activity, comprising:
a battery
a circuit board electronically coupled to the battery;
a casing enclosing the circuit board and the battery;
a first biosensor electronically coupled to the circuit board protruding from a first side of the casing; and
a second biosensor electronically coupled to the circuit board protruding from a second side of the casing.

15. The electronic capsule of claim 14, further comprising an accelerometer, a wireless transmitter, and one or more logic circuits to process electronic input signals from the first biosensor, the second biosensor, and the accelerometer.

16. The electronic capsule of claim 15, wherein the logic circuits store data from the first biosensor, the second biosensor, and the accelerometer, and wherein the logic circuits transmit the data using the wireless transmitter.

17. The electronic capsule of claim 16, wherein the first biosensor and the second biosensor are each configured to detect a plurality of signals caused by electrical activity of the heart during one or more cardiac cycles, and the logic circuits are configured to process the plurality of signals and calculate an RR-interval.

18. The electronic capsule of claim 17, wherein the logic circuits calculate a heart rate variability value and a recovery score.

19. The electronic capsule of claim 15, wherein the wireless transmitter comprises a BLUETOOTH transmitter, a Wi-Fi transmitter, or a GPS transmitter.

20. An activity monitoring device, comprising:
an electronic capsule, comprising:
a battery
a circuit board electronically coupled to the battery;
a casing enclosing the circuit board and the battery;
a finger biosensor electronically coupled to the circuit board protruding from a first side of the casing; and
a wrist biosensor electronically coupled to the circuit board protruding from a second side of the casing; and
one or more wristbands comprising wristbands of varying dimensions, each wristband comprising silicone, rubber, leather, plastic, or metal, and wherein the electronic capsule is configured to interchange between the wristbands of varying dimensions.
